(19) United States
(12) Patent Application Publication
(10) Pub. No.: US 2007/0184490 A1
(43) Pub. Date: Aug. 9, 2007

(54) Neuronal nicotinic receptor ligands and their use

(76) Inventors: Marleen Verlinden, Evanston, IL (US); Michael D. Meyer, Lake Villa, IL (US); Michael W. Decker, Mundelein, IL (US); James P. Sullivan, Deerfield, IL (US); William H. Bunnelle, Mundelein, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/653,553

(22) Filed: Jan. 16, 2007

Related U.S. Application Data
(60) Provisional application No. 60/759,314, filed on Jan. 17, 2006.

(51) Int. Cl. G01N 33/53; A61K 31/4439

(52) U.S. Cl. 435/7.1; 514/337; 514/343

(57) ABSTRACT

The invention relates to neuronal nicotinic receptor ligands, methods of identifying such ligands for neuronal nicotinic receptor modulation, particularly such ligands demonstrating beneficial side effect tolerability, and methods of using such neuronal nicotinic receptor ligands to provide pharmaceutical compositions and products.

This application claims the benefit of U.S. patent application No. 60/759,314, filed Jan. 17, 2006, which is herein incorporated by reference in its entirety.

### BACKGROUND OF THE INVENTION

1. Technical Field

The invention relates to neuronal nicotinic receptor ligands, methods of identifying such ligands for neuronal nicotinic receptor modulation, and methods of using such neuronal nicotinic receptor ligands.

2. Description of Related Technology

Nicotinic acetylcholine receptors (nAChRs) are widely distributed throughout the central (CNS) and peripheral (PNS) nervous systems. Such receptors play an important role in regulating CNS function, particularly by modulating release of a wide range of neurotransmitters, including, but not necessarily limited to acetylcholine, norepinephrine, dopamine, serotonin and GABA. Consequently, nicotinic receptors mediate a very wide range of physiological effects, and have been targeted for therapeutic treatment of disorders relating to cognitive function, learning and memory, neurodegeneration, pain and inflammation, psychosis and sensory gating, mood and emotion, among others.

Many subtypes of the nAChR exist in the CNS and periphery. Each subtype has a different effect on regulating the overall physiological function. Typically, nAChRs are ion channels that are constructed from a pentameric assembly of subunit proteins. At least 12 subunit proteins, α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition (α4)₂(β32)₃ (the α4β2 subtype). Accordingly, various compounds demonstrating activity in neuronal nicotinic receptor (NNR) modulation have been found useful for treating various disorders in which the nicotinic-cholinergic system is implicated, for example disorders or conditions related to cognitive disturbances.

While such NNR ligands have been found effective, their therapeutic activity can be limited due to NNR-mediated side effects. Like plant alkaloid nicotine, certain compounds can interact with various subtypes of the nAChRs. While such compounds may demonstrate many beneficial therapeutic properties, not all of the effects mediated by certain NNR ligands are desirable. For example, nicotine exerts gastrointestinal and cardiovascular side effects that interfere at therapeutic doses, and its addictive nature and acute toxicity are well-known. Ligands that are selective for interaction with only certain subtypes of the nAChR offer potential for achieving beneficial therapeutic effects with an improved margin for safety.

Although various classes of compounds demonstrating nAChR-modulating activity exist, it would be beneficial to provide additional compounds demonstrating the beneficial therapeutic properties of nAChR, and particularly NNR ligands, without the liability of NNR-mediated side effects. In particular, it would be beneficial to provide a method for identifying NNR ligands associated with a low incidence of side effects, particularly NNR-mediated side effects, for example cardiovascular or gastrointestinal irregularities.

### SUMMARY OF THE INVENTION

The invention relates to a method of identifying neuronal nicotinic receptor ligands, and particularly NNR ligands with a significant likelihood of demonstrating low incidence of NNR-mediated side effects or well-tolerated side effects. The method comprises the step of providing a compound demonstrating selectivity for the α4β2 NNR subtype, such compound also demonstrating weak agonist activity at NNRs expressed in vitro. Compounds demonstrating such properties exhibit a significant likelihood of demonstrating beneficial cognitive effects associated with NNR-mediated activities, such as positive effects on cognition. For example, such compounds may demonstrate beneficial therapeutic effect on conditions and disorders characterized by neuropsychological and cognitive dysfunction, for example in Alzheimer's disease, bipolar disorder, schizophrenia, schizoaffective disorder, and other related disorders characterized by neuropsychological and cognitive dysfunction.

In addition, such compounds possess a significant likelihood of retaining beneficial NNR-mediated effects, for example beneficial effects on the neuropsychological system and cognition, while demonstrating a reduced liability for NNR-mediated side effects when compared with NNR ligands that do not demonstrate selectivity for the α4β2 NNR subtype and weak agonist activity at NNRs expressed in vitro. As such, compounds identified by the method of the invention can be associated with a low incidence of cardiovascular and gastrointestinal side effects, which have been confirmed at least in animal models, for example mammalian animal models, such as rodent and primate models, and, can be further confirmed in humans, as demonstrated by study results for a particular NNR ligand, as reported in Appendix A, which is herein incorporated by reference in its entirety.

Moreover, a compound demonstrating selectivity for the α4β2 NNR subtype and weak agonist activity at NNRs, as can be demonstrated by evaluating agonist activity at NNRs expressed in vitro, can be administered to a mammal, or subject, susceptible to or having a condition or disorder wherein modulation of nicotinic receptor activity is of therapeutic benefit to provide a pharmaceutical compound or composition demonstrating such therapeutic benefit. In a clinical study, such compound or composition can be administered to a subject to demonstrate therapeutic benefit for a condition or disorder wherein modulation of nicotinic receptor activity is beneficial. Data can be obtained from the subject and assessed to provide statistical support for therapeutic effect. Such obtained data can be submitted to a regulatory agency having authority to assess and regulate pharmaceutical compounds or products in order to obtain approval to manufacture or market a desired pharmaceutical compound.

The compounds, compositions, methods identifying such compounds, and methods for using the compounds, compositions, or data obtained from administration of such compounds or compositions to a mammal, or subject, is further described herein, for example in the Detailed Description below.

### DETAILED DESCRIPTION OF THE INVENTION

Methods of the Invention

One method of the invention relates to a method of identifying neuronal nicotinic recpeor ligands, particularly neuronal nicotinic agonists demonstrating selective binding for α4β2 neuronal nicotinic receptor subtype and also demonstrating weak agonist activity at neuronal nicotinic receptors expressed in vitro. The method comprises the steps of: 1) assessing a compound for selective binding to α4β2 neuronal nicotinic receptor subtype; 2) assessing a compound for ability to stimulate ion channel flux into a cell expressing α4β2, α3β4, or α3β2 neuronal nicotinic receptor subtypes; 3) and identifying a compound that selectively binds α4β2 neuronal nicotinic receptor subtype and demonstrates weak ability to stimulate ion channel flux into the cell expressing α4β32, α3β4, or α3β2 neuronal nicotinic receptor subtypes.

The compound can be assessed for binding to the α4β2 NNR subtype using various methods. It is understood in the art that one skilled in the art of developing neuronal nicotinic receptor ligands, particularly for pharmaceutical products, would be able to assess selective α4β2 NNR subtype binding in a variety of methods suitable for determining whether a compound binds to α4β2 in a selective manner.

One method for assessing selective α4β2 NNR subtype binding in vitro is via evaluating the ability of a compound to displace [³H]-cytisine from a rat brain membrane preparation. The method can be accomplished under any suitable binding conditions. Examples of suitable binding conditions for [³H]-cytisine binding have been described in the art, for example in at least U.S. Pat. Nos. 5,948,793; 5,914,328; and 6,809,105, the procedures of which are herein incorporated by reference in their entirety. IC₅₀ and Kᵢ values can be determined from data obtained in the [³H]-cytisine binding assay. Preferably, a compound for the method demonstrates less than 30 nM binding affinity, and more preferably less than 15 nM binding affinity, at the [³H]-cytisine binding site.

Alternatively, other methods suitable for assessing the selective binding of a compound for α4β2 can be used. Such methods may vary in preferred binding affinity amounts as determined by the assay. However, one with skill in the art would be able to determine preferred levels for any particular α4β2 selective binding assay of interest taking into account the effect of the compounds selected in suitable in vitro or animal models for evaluating the cognitive enhancing effect of a compound or other NNR-mediated therapeutic benefits and side effects demonstrated by the use of the compound.

The compound can be assessed for ability to stimulate ion channel flux into a cell expressing α4β2, α3β4, or α3β2 neuronal nicotinic receptor subtypes using various methods. It is understood in the art that one skilled in the art of developing neuronal nicotinic receptor ligands, particularly for pharmaceutical products, would be able to assess the ability a compound to stimulate ion channel flux into a cell expressing α4β2, α3β4, or α3β2 neuronal nicotinic receptor subtypes in a variety of methods suitable for determining ion channel flux.

One method for assessing ion channel flux is via activation of ion flux into a cell expressed with recombinant α4β2, α3β4, or α3β2 NNR subtypes. Alternatively, a native cell line that expresses NNRs also can be suitable. The method can be accomplished under any suitable binding conditions. Examples of suitable binding conditions for [³H]-cytisine binding have been described in the art, for example in at least U.S. Pat. Nos. 6,403,575 and 6,133,253, the procedures of which are herein incorporated by reference in their entirety. Data obtained from such ion channel flux assays can be evaluated to determine percent maximal nicotinic response (%), which directly correlates to percent maximal agonist efficacy. Preferably, a compound for the method demonstrates less than 40% maximal agonist efficacy.

Other methods for assessing ion channel flux can be used. Such methods may vary in the percent maximal agonist efficacy as determined by the assay. However, one with skill in the art would be able to determine preferred levels for percent maximal agonist efficacy any particular ion channel flux assay of interest, taking into account the effect of the compounds selected in suitable in vitro or animal models for evaluating the cognitive enhancing effect of a compound or other NNR-mediated therapeutic benefits and side effects demonstrated by the use of the compound.

A neuronal nicotinic receptor ligand demonstrating selective binding for α4β2 neuronal nicotinic receptor subtype and also demonstrating weak agonist activity at neuronal nicotinic receptors expressed in vitro can be identified considering selective α4β2 binding and ion channel flux methods previously described.

Compounds demonstrating such properties exhibit a significant likelihood of demonstrating beneficial cognitive effects associated with NNR-mediated activities, such as positive effects on cognition, including, but not limited to, beneficial therapeutic effect on conditions and disorders characterized by neuropsychological and cognitive dysfunction, for example in Alzheimer's disease, bipolar disorder, schizophrenia, schizoaffective disorder, and other related disorders characterized by neuropsychological and cognitive dysfunction.

In addition, such compounds demonstrate a reduced liability for NNR-mediated side effects when compared with NNR ligands that do not demonstrate selectivity for the α4β2 NNR subtype and weak agonist activity at NNRs expressed in vitro. As such, compounds identified by the method of the invention can be associated with a low incidence of cardiovascular and gastrointestinal side effects. Such compounds can be administered to a mammal, or subject, susceptible to or having a condition or disorder wherein modulation of nicotinic receptor activity is of therapeutic benefit to provide a pharmaceutical compound or composition demonstrating such therapeutic benefit, for example, in a clinical study. Such compound or composition can be administered to a subject to demonstrate therapeutic benefit for a condition or disorder wherein modulation of nicotinic receptor activity is beneficial. Data can be obtained from the subject and assessed to provide statistical support for therapeutic effect. Such obtained data can be submitted to a regulatory agency having authority to assess and regulate pharmaceutical compounds or products in order to obtain approval to manufacture or market a desired pharmaceutical compound. A suitable pharmaceutical compound can be obtained by incorporating the compound in a pharmaceutically acceptable carrier.

Actual dosage levels of compound, or active ingredient, in a pharmaceutical composition of the invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal range from about 0.001 mg/kg body weight to about 1 g/kg body weight. More preferable doses can be in the range of from about 0.10 mg/kg body weight to about 100 mg/kg body weight, and more preferably 1 mg/kg body weight to about 20 mg/kg body weight, and even more preferably 0.05 mg/kg body weight to about 0.5 mg/kg body weight. If desired, the effective daily dose can be divided into multiple doses for purposes of administration. Consequently, single dose compositions may contain such amounts or submultiples thereof to make up the daily dose.

Compounds of the Invention

Compounds suitable for the invention can be any compound that demonstrates selective binding for α4β32 neuronal nicotinic receptor subtype and also demonstrates weak agonist activity at neuronal nicotinic receptors expressed in vitro, unless the compound is a neuronal nicotinic receptor antagonist, for example dihydro-β-erythroidine hydrobromide (DHBE). Examples of suitable compounds are, for example, ABT-089, which is 2-methyl-3-(2-(S)-pyrrolidinylmethoxy)pyridine, having the structure: 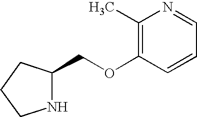
and demonstrating a binding affinity of Kᵢ is 14 nM, which is further described in U.S. Pat. No. 5,948,793, which is herein incorporated by reference in its entirety.

Another suitable compound for the method is, for example, the compound (R,R)-1-(pyridin-3-yl)-octahydro-pyrrolo[3,4-b]pyrrole, having the structure: 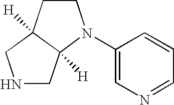
and demonstrating a binding affinity of Kᵢ is 8.8 nM, which is further described in U.S. Pat. No. 6,809,105, which is herein incorporated by reference in its entirety.

Yet another suitable compound for the method is, for example, the compound having the structure: 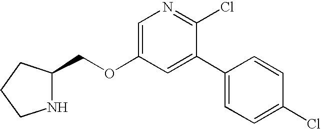
and demonstrating a binding affinity of Kᵢ is 0.04 nM, which is further described in U.S. Pat. No. 6,127,386, which is herein incorporated by reference in its entirety.

Another suitable compound for the method is, for example, the compound having the structure: 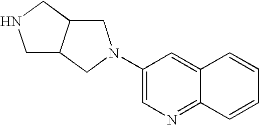
demonstrating the binding affinity of Kᵢ is 1.5 nM, which is further described in U.S. Pat. No. 6,809,105, which is herein incorporated by reference in its entirety.

Such compounds have demonstrated selective α4β2 neuronal nicotinic receptor subtype binding and a weak ability to stimulate ion channel flux in cells expressing α4β2, α3β4, or α3β2 NNR subtypes. Each of the compounds exhibits efficacy at free plasma concentration levels within ten-fold of the neuronal nicotinic binding Kᵢ. Accordingly, it is also contemplated as part of the invention to define target plasma concentration levels for administration of such compound. As such, the invention provides a method for identifying and using compounds to provide maximal efficacy.

Moreover, as demonstrated in the study results attached in the Examples, particularly Example 3, the compound ABT-089 demonstrated efficacy in human clinical studies, as assessed using the Connor's Adult ADHD Rating Scale (CAARS), and was well-tolerated. Methods and materials for assessing the efficacy of ABT-089, a compound demonstrating selective binding for α4β2 neuronal nicotinic receptor subtype and weak agonist activity for neuronal nicotinic receptors expressed in vitro, are described herein in the Examples.

Such human clinical data may be provided to a regulatory authority in order to obtain regulatory authorization. The data may be provided to a regulatory agency having authority to assess or regulate, or both, pharmaceutical compounds or products, or both in order to obtain approval to manufacture or market a desired pharmaceutical compound from the regulatory agency. Such data may be particularly useful where it is related to ABT-089 human clinical data, and more particularly wherein the human clinical data is related to a randomized, double-blind, placebo-controlled multiple dose study. In addition, such human clinical data may be used to provide a pharmaceutical product related to the approval to manufacture or market a desired pharmceutical compound obtained from the regulatory agency. Such data is particularly useful wherein the pharmaceutical product is useful for treating a mammal having a condition where modulation of nicotinic acetylcholine receptor activity is of therapeutic benefit, wherein the condition is Alzheimer's disease, bipolar disorder, schizophrenia, or schizoaffective disorder.

Compositions of the Invention

The invention also provides pharmaceutical compositions comprising a therapeutically effective amount of a desired compound in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N. Y., (1976), p 33 et seq.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of the invention which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable ester,” as used herein, refers to esters of compounds of the invention which hydrolyze in vivo and include those that break down readily in the human body to leave the parent compound or a salt thereof. Examples of pharmaceutically acceptable, non-toxic esters of the invention include C₁-to-C₆ alkyl esters and C₅-to-C₇ cycloalkyl esters, although C₁-to-C₄ alkyl esters are preferred. Esters of the compounds of the invention can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl trifilate, for example with methyl iodide, benzyl iodide, cyclopentyl iodide. They also can be prepared by reaction of the compound with an acid such as hydrochloric acid and an alkylcarboxylic acid such as acetic acid, or with acid and an arylcarboxylic acid such as benzoic acid.

The term “pharmaceutically acceptable amide,” as used herein, refers to non-toxic amides of the invention derived from ammonia, primary C₁-to-C₆ alkyl amines and secondary C₁-to-C₆ dialkyl amines. In the case of secondary amines, the amine can also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of invention can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of the invention, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds.

The compounds, compositions, and methods of the invention will be better understood by reference to the following examples and reference examples, which are intended as an illustration of and not a limitation upon the scope of the invention.

### EXAMPLES

### Determination of Nicotinic Acetylcholine Channel Receptor Binding Potencies

### Example 1

### [³H1-Cytisine Binding Assay

Binding conditions were modified from the procedures described in Pabreza L A, Dhawan, S, Kellar K J, [³H]-Cytisine Binding to Nicotinic Cholinergic Receptors in Brain, Mol. Pharm. 39: 9-12, 1991. Membrane enriched fractions from rat brain minus cerebellum (ABS Inc., Wilmington, DE) were slowly thawed at 4° C., washed and resuspended in 30 volumes of BSS-Tris buffer (120 mM NaCl/5 mM KCI/2 mM CaCl₂/2 mM MgCl₂/50 mM Tris-Cl, pH 7.4, 4° C.). Samples containing 100-200 μg of protein and 0.75 nM [³H]-cytisine (30 Cᵢ/mmol; Perkin Elmer/NEN Life Science Products, Boston, MA) were incubated in a final volume of 500 μL for 75 minutes at 4° C. Seven log-dilution concentrations of each compound were tested in duplicate. Non-specific binding was determined in the presence of 10 μM (−)-nicotine. Bound radioactivity was isolated by vacuum filtration onto prewetted glass fiber filter plates (Millipore, Bedford, MA) using a 96-well filtration apparatus (Packard Instruments, Meriden, CT) and were then rapidly rinsed with 2 mL of ice-cold BSS buffer (120 mM NaCl/5 mM KCl/2 mM CaCl₂/2 mM MgCl₂). Packard MicroScint-20® scintillation cocktail (40 μL) was added to each well and radioactivity determined using a Packard TopCount® instrument. The IC₅₀ values were determined by nonlinear regression in Microsoft Excel® software. Kᵢ values were calculated from the IC₅₀s using the Cheng-Prusoff equation, where Kᵢ=IC₅₀/1+[Ligand]/K_{D}].

### Determination of Nicotinic Acetylcholine Channel Receptor Channel Ion Flux

### Example 2

### IMR-32 Assay

Cells of IMR-32 human neuroblastoma clonal cell line (ATCC, Rockville, Md., USA) were maintained in a log phase of growth according to established procedures. Experimental cells were seeded at a density of 500,000 cells/mL into a 24-well tissue culture dish. Plated cells were allowed to proliferate for at least 48 hours before loading with 2 μCᵢ/mL of ⁸⁶Rb⁺ (35 Cᵢ/mmol) overnight at 37° C. The ⁸⁶Rb⁺ efflux assays were performed according to previously published protocols (Lukas, R. J., J. Pharmacol. Exp. Ther., 265, 294-302, 1993) except serum-free Dulbecco's Modified Eagle's medium was used during the ⁸⁶Rb⁺ loading, rinsing, and agonist-induced efflux steps. Data reflect the activation of ⁸⁶Rb⁺ flux at a concentration of 1 μM, and reflect the response as a percentage of the maximum response elicited by (S)-nicotine. The data are interpreted such that the larger the response, the more potent is the activation of peripheral ganglionic receptors, which is further interpreted to suggest that, in vivo, a more potent contribution to undesired effects will occur, for example on the cardiovascular or gastrointestinal systems, or both.

### Pilot Study of A Neuronal Nicotinic Receptor Partial Agonist For the Treatment of ADHD in Adults

### Example 3

A pilot study was designed to evaluate ABT-089, a neuronal nicotinic receptor (NNR) partial agonist, as treatment for adult attention-deficit hyperactivity disorder (ADHD). Method: Adults with ADHD received placebo, 2mg, 4mg, or 20mg of ABT-089 for two weeks each in a randomized, double blind, placebo-controlled 4×4 Latin square design for a total of 8 weeks. In addition to the primary outcome, the Conner's Adult ADHD Rating Scale (CAARS), secondary rating scales, neuropsychological, and safety assessments were completed. Results: A total of 11 adults with well-characterized ADHD completed this crossover study. ABT-089 was superior to placebo for the CAARS Total Symptom Score, which was the primary endpoint (placebo: 38.0±−1.9; 2 mg bid: 32.2±−1.9, one-tail p=0.021; 4 mg bid: 33.2±−1.9, p=0.047; 20 mg bid: 33.5±−1.9, p=0.056). ABT-089 was also superior to placebo for the CAARS ADHD index and Hyperactive/Impulsive scores and the Clinical Global Impression-ADHD Severity score. On the clinical efficacy endpoints, CAARS Total Symptom Score and CAARS Hyperactive/Impulsive score, a shallow inverted V-shaped dose-response curve was observed. However, the dose-response curve for attention and memory effects as measured by computerized cognitive testing seemed dose-linear. No clinically meaningful findings in safety assessments or side effect profile were observed. Conclusions: Data from this pilot study suggest that ABT-089 may be effective in treating adult ADHD and is well tolerated. Based on these promising results, larger parallel-group ABT-089 studies of longer duration are warranted.

### INTRODUCTION

Attention deficit-hyperactivity disorder (ADHD) is characterized by core symptoms of hyperactivity, inattentiveness, and impulsivity. Its prevalence in school-age children is estimated at 6-8% worldwide (Faraone et al 2003), with symptoms persisting into adulthood in approximately 50% of individuals with childhood onset (Barkley et al 2002; Wilens et al 2004). Recent epidemiological data suggests that ADHD occurs in approximately 4.7% of adults in the US (Kessler et al in press). The aggregate data also support that ADHD in adults shares many phenotypic and genotypic similarities with the childhood form of the disorder (Faraone et al 2005; Spencer 2004). Moreover, because of the pharmacological similarity in response across the lifespan (Spencer 2004) and ethical considerations of exposing youth to novel compounds, adults with ADHD have been used increasingly for early phase pharmacological trials.

ADHD in adults is associated with academic, employment, and marital difficulties, as well as comorbid psychiatric disorders such as substance abuse, depression, anxiety, and personality disorders (Barkley 2002; Biederman 2004; Wilens et al 1995). Moreover, cost-of-illness data in untreated adults with ADHD vastly exceeds that in matched adults with ADHD—both for medical and societal expenses (Secnik et al 2005). Given the high level of impairment in functioning and dysfunction in quality of life, adults with ADHD require treatment for their ADHD.

Currently, the treatment of ADHD in adults is largely predicated upon use of both stimulant and nonstimulant medications, as well as adjunctive structured psychotherapies (Saften et al 2004). Despite the availability of both Food and Drug Administration (FDA) approved and other agents for ADHD, a number of individuals either cannot tolerate, or do not respond to existing compounds necessitating the development of alternative agents with novel mechanisms of action.

Increasing interest has been focused on the role of the nicotinic-cholinergic system in cognitive disturbances including ADHD. In addition to cigarette smoking being overrepresented in adolescents and adults with ADHD, nicotine and related analogs have been shown to have efficacy in treating ADHD (Conners 1996; Levin et al 1996; Wilens et al 1999).

For example, ABT-418, a neuronal nicotinic receptor (NNR) agonist administered transdermally, was previously shown in a controlled clinical trial in 32 adults to be effective in treating ADHD in general, and attentional/cognitive deficits in particular (Wilens et al 1999). More recently, an oral form of a NNRpartial agonist, ABT-089, has become available for human testing. ABT-089 very selectively binds to human α4 β2 NNRs in vitro and has weak agonist activity at NNRs expressed in vitro. ABT-089 has been shown in rodent and primate animal models to improve attention, learning, and memory deficits. The dose-response curve was U-shaped, with efficacy associated with plasma levels of 5-15 ng/mL (Rueter et al 2004). Similar findings have been observed in a Phase 1 multiple dose human study. In this multiple dose study, Simple Reaction Time, a measure of attention, was significantly improved with ABT-089 over a range of 5 to 40 mg twice daily, as compared to placebo [M02-41 1, data on file at Abbott Laboratories]. ABT-089 was well tolerated over this dose range. Given the effects of this nicotinic analog on cognition impairments, and given the higb degrees of cognitive dysfunction in ADHD (particularly adolescents and adults) (Biederman et a12000; Millstein et all 997), the use of ABT-089 therapeutically for ADHD is compelling.

Reported herein are the results of a randomized, double blind, placebo-controlled crossover pilot study of ABT-089 in the treatment of adults with ADHD. Our objective was to compare the safety and efficacy of 2 mg, 4 mg, and 20 mg of ABT-089 twice daily to placebo in adults with ADHD. We hypothesized that the ABT-089 phases of the study, compared to placebo condition, would be associated with improvement in the core symptoms of ADHD.

This study was designed as an exploratory, signal-detection, Phase IIa study to provide proof-of-concept for this novel compound prior to embarking on a larger scale Phase lib program. As such, a cost-efficient design was selected in which a relatively small number of subjects would be studied at a small number of highly experienced study sites. One method chosen for keeping the number of subjects small without losing statistical power was to employ a one-tailed test to test the hypothesis that drug is better than placebo, thus reducing the number of subjects by 20%. In addition, a crossover design was selected rather than a parallel group design, thus further reducing the number of subjects to be studied approximately tenfold compared to a conventional 4-arm parallel dose-ranging study of similar statistical power with two-tailed testing. Subjects received placebo and three doses of ABT-089. A broad range of doses was selected to maximize the probability of signal detection. A relatively rapid onset of effect was expected (˜1-2 hours, data on file at Abbott), so the duration of dosing was limited to two weeks per treatment; however, the design ensured that each subject received uninterrupted exposure to study drug for four to six weeks.

The study was stopped before all subjects completed the trial. A total of 61 subjects had enrolled and 11 had completed the study. This publication focuses on the results of the 11 subjects who completed the entire crossover trial. The 50 remaining patients had only partial data, mostly confined to the first two weeks of the study.

Methods and Materials

Subjects

Subjects between 18 and 60 years old who met the DSM-IV-TR criteria for ADHD as assessed by clinical interview and confirmed by the Washington University in St. Louis Kiddie Schedule for Affective Disorders and Schizophrenia (WASH-U-KSADS) diagnostic criteria for ADHD (Orvaschel 1985) (Geller et al 1998) were eligible for inclusion in the study. Furthermore, a score of ≧2 on at least six of nine items in at least one of the subscales of the Conner's Adult ADHD Rating Scale (CAARS) (Conners et al 1999) at screening and Day 1 and a score of ≧4 (i.e., at least moderate severity) on the Clinical Global Impressions-ADHD Severity (CGI-ADHD-S) test (Guy 1976) at screening were required. Exclusion criteria consisted of: smoker or user of nicotine product(s) in the three months prior to enrollment; clinically significant chronic medical conditions; current diagnosis or history of schizoaffective or bipolar disorder, obsessive-compulsive disorder, schizophrenia, or other psychotic disorder; current depression requiring treatment; serious homicidal or suicidal ideation; abnormal baseline laboratory values; drug or alcohol abuse/dependence within the last three months; current use of psychotropics or stimulants; and pregnant or lactating women. The following institutional review boards at Quorum IRB in Seattle, Wash., The Human Research Committee at the Lawrence House in Boston, Mass., the Institutional Board of Research Associates at the NYU School of Medicine in New York, N.Y., the New York Campus, Va. NY Harbor Healthcare Systems Subcommittee for human subjects, research and development subcommittee, and research safety subcommittee in New York, N.Y., the University of Vermont Committee on Human Research in Burlington, Vt., the Scientific Advisory Committee of Burlington, Vt., and the University of Chicago Hospitals IRB in Chicago, Ill. approved the study protocol, and all subjects provided written, informed consent.

Design

Following an initial two-week medication washout and screening period, subjects entered an eight-week double-blind treatment period. Eligible subjects were randomized (according to a central computer-generated randomization schedule) to one of four treatment sequences in which they received ABT-089 2 mg, 4 mg, and 20 mg as well as matching placebo, each twice daily (30 minutes before breakfast and eight hours later) for two weeks, with no washout between the treatments. A study design schematic is provided below in Table 1.

Assessments

Raters for the CAARS were trained and certified prior to the start of the study. Investigator-administered CAARS, CGI-ADHD-S, Hamilton Anxiety Scale (HAM-A) (Hamilton 1959), and Hamilton Depression Scale (HAM-D) (Hamilton 1960) were administered to subjects at baseline and at the completion of each treatment period (Days 14, 28, 42, and 56). CMRS, HAM-A, and HAM-D ratings were based on the previous seven days. After being trained, subjects completed a computerized cognitive assessment battery (Simpson et al 1989), which was amended to include the Conner's Continuous Performance Test (Conners 1995) and the Stroop Color Word Test (Jensen and Rohwer 1966), at baseline and at the completion of each treatment period. Attentional tasks (simple and choice reaction time, digit vigilance), selective attention (Conner's CPT), working memory tasks (numeric and spatial working memory, rapid visual information processing), episodic secondary memory (immediate and delayed word recall, word and picture recognition), motor control tasks (tracking), and executive function (the Stroop effect) were among the items assessed.

Blood samples for pharmacokinetic analysis were taken at 0, 1, 2, 4, and 8 hours on Day 1 and at approximately 2 hours post-dose on the last day of each dosing period. The blood samples were immediately stored at 4° C. or below. The blood samples were centrifuged within one hour of collection using a refrigerated centrifuge to separate the plasma. The plasma samples were transferred using plastic pipettes into plastic vials. The plasma samples were frozen at −20° C. within one hour from centrifugation and remained frozen until shipped to Abbott Laboratories for analysis.

Safety was evaluated by spontaneous report of adverse events; in addition, laboratory data (hematology, chemistry, urinalysis), vital signs, and electrocardiograms (BCGs) were completed at baseline and at the end of each treatment period.

Statistics

The primary efficacy endpoint was the CAARS total ADHD symptom score (sum of Inattention and Hyperactivity/Impulsivity scores) obtained on the last day of each treatment period. Treatment differences were assessed using an analysis of variance (ANOV A) model with fixed terms fitted for treatment, period, and sequence and a random effect for subjects-within-sequence. Treatment differences for secondary efficacy measures (ADHD Index, CAARS Hyperactive/impulsive score, CAARS Inattentive score, CGI-ADHD-S, HAM-A, HAM-D), and the computerized cognitive assessment battery) and mean laboratory, vital signs, and electrocardiogram (ECG) data obtained at the end of each treatment period were also evaluated by ANOV A. Within the framework of the ANOV A model, each dose of ABT-089 was compared to placebo. There were no corrections for multiple comparisons in this proof-of-concept study. Statistical tests of efficacy were one-sided; p-values ≦0.050 were considered statistically significant, and those between 0.051 and 0.010 indicated a statistical trend.

The one-sided test was chosen a priori because the conduct of a full-sized parallel group dose-ranging trial with two-tailed testing would be predicated on at least demonstrating improvement with ABT-089 compared to placebo. The assumed effect size of 0.37 is consistent with results from the atomoxetine multicenter trials in adults with ADHD after two to ten weeks of treatment (Michelson et al 2003). A within-subject correlation of 0.5 was assumed as a lower bound to correlations from published test/retest reliability results for the scale.

A Williams design, a type of Latin square design, was adopted for this study (Senn 1993). A Williams design is a special case of a crossover design in which each treatment precedes all other treatments an equal number of times, and allows for an assessment of unequal carryover effects. The power of this design relies on all subjects completing all four treatment periods. For an effect size of 0.37 and a within-subject correlation of 0.5, a sample size of 48 subjects completing all treatments would detect superiority of an ABT-089 dose relative to placebo with 80% power in a one-tailed test with alpha=0.05. A parallel group Phase lb dose ranging trial with two-tailed testing requires 130 subjects per treatment group, for a total of 520 subjects.

In order for subjects to not be without active treatment more than two weeks during the study (i.e., placebo during one of the four study treatment periods), and to allow continuous treatment with “active” drug for at least four weeks, there was no washout interval between doses of consecutive periods. Performing evaluations at the end of the two weeks of treatment should have minimized, if not eliminated, carryover. If evidence of an unequal carryover existed, the sequences chosen for the study would have allowed for adjustment in the analyses.

Empirical effect sizes were calculated for the CAARS total score as the mean difference for each ABT-089 dose versus placebo divided by the standard deviation of the difference scores.

Results

Demographics and Disease History

A total of 61 subjects were enrolled and 11 completed the study before it was prematurely terminated pending additional preclinical data. Only one subject prematurely discontinued study participation due to an adverse event, dizziness (on Study Day 2, while taking ABT-089 2 mg), which the investigator thought may have been related to study drug or concomitant use of alcohol and illicit drugs acutely. One subject withdrew consent during the study, one subject was lost to follow-up, and the remaining 47 subjects discontinued when the sponsor stopped the study prematurely. Most of these subjects had completed only 5 to 14 days of treatment and hence, were not included in the analyses. Given that the statistical power of the Williams crossover design requires subjects to complete all treatment periods, efficacy analyses were conducted using the dataset of the 11 subjects who completed the study, which included six males and eight Caucasians, with a mean (±SD) age of 32.0 (10.15) years. Five subjects had a first degree relative with ADHD. Three subjects had at least one lifetime comorbid psychiatric illness, all three of whom reported depression.

At baseline, six subjects met the DSM-IV criteria for the CAARS Inattentive subtype, five met the criteria for the combined subtype, and none met the criteria for the CMRS Hyperactive/impulsive subtype. At baseline, mean CAARS scores were 39.1 (8.08) for total score, 22.6 (3.14) for Inattentive sub scale and 16.5 (6.09) for Hyperactive/impulsive sub scale, and 25.4 (4.74) for ADHD Index. The subjects were neither highly anxious (HAM-A=7.1±5.15) nor depressed (HAM-D=5.5±3.50). Baseline characteristics of subjects who completed the trial are provided below in Table 2. They were impaired on some computerized cognitive assessments in comparison to age-matched healthy controls, reflected in impaired reaction times within the individual Attentional tasks.

Treatment Effects

On the last treatment day, a statistically significant treatment effect (vs. placebo) on the CAARS total symptom score was observed for both ABT-089 2 mg and 4 mg twice daily doses and the response approached the level of statistical significance (p=0.056) for ABT-089 20 mg twice daily as reported below in Table 3. The effect size was 0.92, 0.76, and 0.71 for the 2 mg, 4 mg, and 20 mg twice daily doses, respectively. When the data were analyzed by CAARS subscales, statistically significant improvements were observed for the ADHD Index at all dose levels after only two weeks of treatment (˜17% improvement vs. placebo) and the Hyperactive/impulsive score in the ABT-089 2 mg and 4 mg doses (˜20% improvement vs. placebo). A trend for improvement was noted on the Inattentive score of the CAARS for the 2 mg and 20 mg doses, with improvement versus placebo scores of approximately 11%. The dose response curve had a shallow inverted U shape, similar to animal data, for both CAARS total symptom score and CAARS Hyperactive/impulsive subscales. No dose response was observed for the CAARS Inattentive subscale, however.

For the primary endpoint, the response to placebo was similar across all four treatment periods, demonstrating absence of learning effects or period effects (Period I: [n=3] 36.7, Period 2: [n=4] 38.5, Period 3: [n=2] 40.5, Period 4: [n=2] 35.5). In the active doses, treatment effects appeared to be greater when treatment duration was longer.

For the CGI-ADHD-S, a treatment difference favoring ABT-089 over placebo was observed following the 2 mg (p=0.031) and 4 mg (p=0.093) doses, but not following the 20 mg dose (p=0.1 12). The mean score following placebo treatment was 4.47 (0.20), compared to 3.92 (0.20) following the 2 mg dose, 4.09 (0.20) following the 4 mg dose, and 4.12 (0.20) following the 20 mg dose. ABT-089 had no effect on HAM-A or HAM-D scores.

Cognitive Assessment.

Results of computerized cognitive assessments in this small sample indicated that the ABT-089 dose-response curve for attention and memory effects seemed to be dose-linear. For numeric working memory sensitivity index, there was a trend favoring ABT-089 20 mg (0.923±0.022) compared to placebo (0.882±0.022; P=0.091). For spatial working memory, statistically significant improvement in sensitivity index was observed with ABT-089 20 mg (0.966±0.031 vs placebo: 0.892±0.031, p=0.021) and a trend with 2 mg (0.93 7±0.031, P=0.074) and 4 mg (0.946±0.031, p=0.052). For information processing, there was a trend favoring ABT-089 20 mg based on speed (516.3±12.7 vs placebo: 544.1±12.7, p=0.065), but no differences between doses for percent of targets detected or number of false alarms. In a measure of selective attention (Continuous Performance Test), statistically significant improvement was observed at all ABT-089 dose levels for number of commission errors, which occur when a response is made to a non-target stimulus. Other treatment dose differences favoring ABT-089 from the Continuous Performance Test included: 2 mg (0.56±0.27 vs placebo: −0.03±0.27, p=0.066), 4 mg (0.68±0.27, p=0.037), and 20 mg (0.69±0.27, p=0.035) for Attentiveness. For measures of attention (reaction time, vigilance), episodic secondary memory, and executive function (assessed by the Stroop Effect), there were no meaningful differences between ABT-089 and placebo.

Pharmacokinetics

Due to limited pharmacokinetic sampling timepoints, trough and average concentrations were estimated using a non-linear mixed-effect pharmacokinetic modeling approach with NONMEM software Version V). Following the 2 and 4 mg twice daily doses, values were within the target range of 5-15 ng/mL as shown in Table 4, below.

Tolerability and Safety

ABT-089, in a dosage between 2 mg and 20 mg twice daily, was well tolerated by the II adult ADHD subjects who completed this study. There were no serious adverse events. There were no clinically meaningful trends in types of adverse events nor any temporal or dose relationship to drug administration. Most adverse events were considered mild or moderate in severity. The most commonly reported treatment-related events (more than one subject during treatment in anyone period) were headache, somnolence, pain (arm pain and toothache), increased appetite and nervousness as shown in Table 5, below. Only one subject experienced nausea at the 4 mg dose, which did not occur at 20 mg or 2 mg and the same subject reported diarrhea while taking 4 mg and 20 mg.

There were no clinically meaningful findings or dose-related trends for laboratory findings, vital signs or ECG during the study. Results from safety evaluations for the 61 subjects who were randomized into this trial, and of whom the majority had been treated for between 5 and 14 days, were consistent with those reported for the 11 completers.

Summary of Results

In this small, pilot, randomized, double-blind, placebo-controlled crossover proof-of-concept study of 11 adult subjects with ADHD, treatment with ABT-089 was well tolerated over a tenfold dose range. Despite the short duration of exposure, the trial results yielded a clear signal of efficacy in improving the symptoms of ADHD when assessed by the investigator-administered CAARS. Both hyperactive/impulsive symptoms as well as inattentive symptoms responded to ABT-089; however, in this study, the effect on hyperactive/impulsive symptoms was numerically greater than the effect on inattentive symptoms. In general, clinical improvements were seen at the lower two doses (2 mg and 4 mg twice daily) on both the primary and secondary outcome measures. In this study, the CGI-ADHD-S showed modest yet significant improvement over placebo at the ABT-089 2 mg dose, supporting the finding on the CAARS of efficacy at lower doses. However, the dose-response curve for attention and memory effects as measured by computerized cognitive testing seemed to be dose-linear. That efficacy was detected after only two weeks of treatment at each dose level compared to placebo suggests a rapid onset of efficacy of ABT-089 for ADHD. Interestingly, the response to placebo was similar in all four treatment periods, indicating the absence of carryover effects.

To be able to detect these promising results in so few subjects following a relatively brief treatment duration gives credence to the use of a crossover design in ADHD, especially for a compound with relatively rapid onset of action, like ABT-089. The crossover design is particularly well-suited to a proof-of-concept study when the symptoms of the disorder under study are stable over time. While appropriate for exploratory studies, such a design would not necessarily be appropriate for a confirmatory study. Crossover designs have been used in prior adult ADHD proof-of-concept studies with atomoxetine (Spencer et all 998), Adderall (Spencer et al2001), and ABT-418 (Wilens et al 1999).

One of the dilemmas of early phase studies is the determination of the optimal dose(s) for a disorder. The doses evaluated for this study were selected on the basis of expectations from pharmacokinetic modeling that 4 mg administered twice daily, given at an 8-hour interval, would maintain population steady-state plasma concentrations in the range of 5-15 ng/mL, uninterruptedly, for approximately 20 hours after the morning dose, in more than 70% of subjects. The targeted range of 5-15 ng/mL was derived from animal experiments (Decker et al 1997) and the 2 mg and 20 mg twice daily doses were employed to test efficacy below and above the expected efficacious plasma levels, respectively. Plasma levels following the two lower doses in this study were within the targeted range, thus confirming our predictions from animal models.

In this pilot study, ABT-089 was associated with improvements in ADHD relative to placebo. Given that ABT-089 has selectivity for the α4 β2 receptor subtype, it is possible that these effects are mediated by this NNR subtype. The results of this study are consistent with a growing literature supporting that direct NNR stimulation is associated with improvements in cognitive deficits of ADHD and related disorders (Newhouse et al 2004). Compounds such as NNRs, with positive effects on cognition, may be beneficial therapeutically in a myriad of disorders characterized by massive neuropsychological and cognitive dysfunction including Alzheimer's disease, bipolar disorder, schizophrenia, schizoaffective disorder, and other related disorders. Clearly, more work evaluating the role of these agents in a broad spectrum of psychiatric disorders with known cognitive disturbance is necessary.

In relation to ADHD, the current results are similar to those by Levin and Conners who reported that a very brief trial of the nicotine patch was effective in ADHD adults (Levin et al 1996). Similarly, a related nicotinic agonist was shown to be useful clinically for ADHD in adults (Wilens et al 1999). These aggregate data further support a growing and well documented link between nicotinic receptor activity and ADHD.

ABT-089 was well tolerated. In the current study, there were no dose-limiting side effects or reports of withdrawal symptomatology. There were no clinically meaningful cardiovascular or other laboratory abnormalities during the study; yet, our ability to detect infrequent and idiosyncratic reactions is limited by the small number of subjects and short-term duration of the treatment conditions and overall study. In addition, as is standard practice in many clinical trials, spontaneous reporting of side effects was used in this study. The use of a structured side effect rating scale may have elicited more adverse events.

There are a number of limitations in the current study. Because of the nature of the study, a homogenous study population was selected that may not generalize to typical adults with ADHD. For example, subjects with significant medical histories and smokers were excluded. In addition, there was a small group of subjects in the study. The study was intended to complete 48 subjects, and although 61 subjects were randomized, the study was prematurely terminated when only 11 subjects had completed all four treatment periods and most others had completed 5-14 days of treatment. In addition, most patients in this study were from one of the study sites, therefore the substantial effect size observed in this study may not repeat in a multicenter trial due to intersite variability. Even though the design ensured that each subject received uninterrupted exposure to study drug for four to six weeks, another limitation was the relatively short exposure to treatment during each period. Two final limitations of the study were the use of one-sided testing for efficacy assessments and lack of adjustment for multiple comparisons, both of which increased the chances of finding a positive effect of ABT-089. The one-sided tests precluded the detection of a negative effect. However, in a proof of concept study, a decision to proceed with development requires that the treatment of interest have an advantage over placebo; findings of no detectable positive effect or of a negative effect on efficacy point to the same conclusion: lack of efficacy.

Despite the limitations presented above, these pilot data show that ABT-089 appears to have efficacy in the treatment of ADHD. Likewise, ABT-089 appeared to be tolerated in this limited number of subjects over a relatively wide dose range (ten-fold). Given the positive findings in this small group of subjects, larger, parallel design dose ranging studies with ABT-089 are warranted, and, given the shallow inverted-U shape of the dose-response curve for clinical endpoints, these studies should focus on the lower end of the dose range that was explored in this study.

TABLE 1
Study Design Schematic

| | Sequence | N | Period 1 | Period 2 | Period 3 | Period 4 |
|---|---|---|---|---|---|---|
| Screening Days - | 1 | 3 | Placebo | 2 mg ABT-089 | 20 mg ABT-089 | 4 mg ABT-089 |
| 14 through −1 | 2 | 2 | 2 mg ABT-089 | 4 mg ABT-089 | Placebo | 20 mg ABT-089 |
| Randomization | 3 | 2 | 4 mg ABT-089 | 20 mg ABT-089 | 2 mg ABT-089 | Placebo |
| on Day 1 | 4 | 4 | 20 mg ABT-089 | Placebo | 4 mg ABT-089 | 2 mg ABT-089 |
| | | | 2 weeks | 2 weeks | 2 weeks | 2 weeks |

Note:
Study drug dosing was twice daily

TABLE 2
Baseline Characteristics of Subjects Who Completed the Trial

| | N = 11 | |
|---|---|---|
| Gender N (%) | | |
| Male | 6 | (54.5%) |
| Female | 5 | (45.5%) |
| Age Mean (SD) | 32.0 | (10.15) |
| ADHD Subtype N (%) | | |
| Combined | 5 | (45.5%) |
| Inattentive | 6 | (54.5%) |
| Hyperactive/Impulsive | 0 | |
| CAARS-INV Mean (SD) | | |
| Total ADHD Symptom Score | 39.1 | (8.08) |
| Inattentive | 22.6 | (3.14) |
| Hyperactive/Impulsive | 16.5 | (6.09) |
| ADHD Index | 25.4 | (4.74) |
| CGI-ADHD-S Mean (SD) | 4.5 | (0.52) |
| HAM-A Mean (SD) | 7.1 | (5.15) |
| HAM-D, 21-Item Mean (SD) | 5.5 | (3.50) |

ADHD = attention deficit hyperactivity disorder; CAARS-INV = Conner's Adult ADHD Rating Scale Investigator Total ADHD Symptom Score; CGI-ADHD-S = Clinical Global Impressions of Severity of ADHD; HAM-A = Hamilton Anxiety Scale; HAM-D = Hamilton Depression Scale

TABLE 3
Least Square Mean (±SE) Conner's Adult ADHD Rating Scale (CAARS) Score (N = 11)

| | CAARS Total | | CAARS Subscales | | | | | |
|---|---|---|---|---|---|---|---|---|
| | | P-value | | P-value | | P-value | | P-value |
| Treatment | | vs. | | vs. | Hyperactive/ | vs. | ADHD | vs. |
| Regimen | Total | Placebo | Inattentive | Placebo | Impulsive | Placebo | Index | Placebo |
| Placebo | 38.0 (1.9) | | 20.7 (1.2) | | 17.3 (0.9) | | 23.7 (1.2) | |
| ABT-089 | 32.2 (1.9) | 0.021 | 18.3 (1.2) | 0.088 | 13.8 (0.9) | 0.005 | 19.7 (1.2) | 0.014 |
| 2 mg bid | | | | | | | | |
| ABT-089 | 33.2 (1.9) | 0.047 | 18.7 (1.2) | 0.128 | 14.5 (0.9) | 0.018 | 19.4 (1.2) | 0.009 |
| 4 mg bid | | | | | | | | |
| ABT-089 | 33.5 (1.9) | 0.056 | 18.1 (1.2) | 0.070 | 15.4 (0.9) | 0.069 | 19.9 (1.2) | 0.017 |
| 20 mg bid | | | | | | | | |

Note:
Least-square means and one-sided P-values from ANOVA analyses with factors for treatment sequence, subject (sequence), period and treatment.

TABLE 4
Population PK Model-Predicted Trough (C_{trough}) and Average (C_{avg})
Plasma Concentrations at Steady State

| | C_{trough} (ng/mL) | | C_{avg} (ng/mL) | |
|---|---|---|---|---|
| Regimen | Mean (±SD) | Range | Mean (±SD) | Range |
| ABT-089 | 1.85 (0.79) | 0.78-3.56 | 4.47 (1.05) | 2.97-6.17 |
| 2 mg bid | | | | |
| ABT-089 | 3.69 (1.59) | 1.56-7.11 | 8.94 (2.09) | 5.93-12.33 |
| 4 mg bid | | | | |
| ABT-089 | 18.46 (7.95) | 7.82-35.56 | 44.7 (10.46) | 29.67-61.66 |
| 20 mg bid | | | | |

PK = Pharmacokinetics

TABLE 5
Adverse Events Reported by at Least Two Subjects
Who Completed the Trial (N = 11)

| | | ABT-089 | ABT-089 | ABT-089 | |
|---|---|---|---|---|---|
| COSTART Term | Placebo | 2 mg bid | 4 mg bid | 20 mg bid | Overall |
| Headache | 0 | 3 | 0 | 2 | 5 |
| Somnolence | 1 | 0 | 0 | 2 | 3 |
| Pain | 0 | 0 | 0 | 2 | 2 |
| Increased appetite | 1 | 0 | 0 | I | 2 |
| Nervousness | 0 | 1 | 0 | 1 | 2 |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art.

### REFERENCES

Complete citations of the references mentioned herein are provided below. References cited below are herein incorporated by reference.
Barkley R (2002): Major life activity and health outcomes associated with attention-deficit hyperactivity disorder. J Clin Psychiatry 63: 10-15.
Barkley R A, Fischer M, Smallish L, Fletcher K (2002): The persistence of Attention-Deficity/Hyperactivity Disorder Into young adulthood as a function of reporting source and definition of disorder. J Abnorm Psychol 111:279-289.
Biederman J (2004): Impact of comorbidity in adults with attention-deficit/hyperactivity disorder. J Clin Psychiatry 65:3-7.
Biedennan J, Mick E, Faraone S V (2000): Age-dependent decline of symptoms of attention deficit hyperactivity disorder: Impact of remission definition and symptom type. Am J Psychiatry 157:816-818.
Conners C, Levin, E D, Sparron, E, Hinton, S C, Erhardt D, Meek, W H, Rose, J E, March, J (1996): Nicotine and attention deficit hyperactivity disorder (ADHD). Psychopharmocol Bull 32:67-73.
Conners C K (1995): The Continuous Performance Test. Toronto, Canada: Multihealth Systems.
Conners C K, Erhardt D, Sparrow E (1999): Conners' Adult ADHD Rating Scales (CAARS).North Tonawanda, NY: Multi-Health Systems.
Decker M W, Bannon A W, Curson P, Gunther K L, Brioni J D, Holladay M W, et al (1997): ABT-089 [2-Methyl-3-(2-(S)-pyrrolidinylmethoxy)pyridine dihydrochloride]: II. A Novel Cholinergic Channel Modulator with Effects on Cognitive Performance in Rats and Monkeys. J Pharmacol Exp Ther283:247-258.
Faraone S V, Pedis R H, Doyle A E, Smoller J W, Goralnick J J, Holmgren M A, et al (2005): Molecular genetics of attention-deficit/hyperactivity disorder. Biol Psychiatry 57: 1313-23.
Faraone S V, Sergeant J, Gillberg C, Biederman J (2003): The Worldwide Prevalence of ADHD: Is it an American Condition? World Psychiatry 2: 104-113.
Geller B, Warner K, Williams M, Zimennan B (1998): Prepubertal and young adolescent bipolarity versus ADHD: assessment and validity using the W ASH-U-KSADS, CBCL and TRF. J Affect Disord 51:93-100.
Guy W (1976): Clinical Global Impressions in Early Clinical Drug Evaluation Unit (ECDEU) Assessment Manual for Psychopharmacology, Vol DHEW Publication No. ADM 76-338. Rockville, Md.: National Institute of Mental Health, pp 218-222.
Hamilton M (1959): The assessment of anxiety states by rating. Br J Med Psychol 32:50-55.
Hamilton M (1960): A rating scale for depression. J Neurol Neurosurg Psychiatry 23:56-62.
Jensen A R, Rohwer W D (1966): The stroop color word test: A review. Acta Psychol 25:36-93.
Kessler R C, Adler L, Barkley R, Biederman J, Conners C K, Demler 0, et al (in press): The prevalence and correlates of adult ADHD in the United States: Results from the national comorbidity survey replication. Am J Psychiatry.
Levin E D, Conners C Sparrow E, Hinton S C, Erhardt D, Meck W H, et at (1996): Nicotine effects on adults with attention-deficit/hyperactivity disorder. Psychopharmacology 123:55-63.
Michelson D, Ad1er L, Spencer T, Reimherr FW, West SA, Allen Al, et al (2003): Atomoxetine in adults with ADHD: two randomized, placebo-controlled studies. Biol Psychiatry 53: 112-20.
Millstein R, Wilens T, Biedennan J, Spencer T (1997): Presenting ADHD symptoms and subtypes in clinically referred adults with ADHD. J Attent Disord 2:159-166.
Newhouse P A, Potter A, Singh A (2004): Effects of nicotinic stimulation on cognitive performance. Curr Opin Pharmacol 4:36-46.
Orvaschel H (1985): Psychiatric interviews suitable for use in research with children and adolescents. Psychopharmacol Bull 21 :737-745.
Rueter L E, Anderson D J, Briggs C A, Donnelly-Roberts D L, Gintant G A, Gopalakrishnan M, et al (2004): ABT-089: pharmacological properties of a neuronal nicotinic acetylcholine receptor agonist for the potential treatment of cognitive disorders. CNS Drug Rev 10: 167-82.
Safren S A, Sprich S, Chulvick S, Otto M W (2004): Psychosocial treatments for adults with attention-deficit/hyperactivity disorder. Psychiatr Clin North Am 27:349-60.
Secnik K, Swensen A, Lage M J (2005): Comorbidities and costs of adult patients diagnosed with attention-deficit hyperactivity disorder. Pharmacoeconomics 23:93-102.
Senn S (1993): Crossover Trials in Clinical Research. Chicester, West Sussex England: John Wiley & Sons LTD.
Simpson P M, Wesnes K A, Christmas L (1989): A computerized system for assessment of drug-induced performance changes in young, elderly or demented populations. Br J Clin Pharmacol27:7111P-712P.
Spencer T (2004): ADHD treatment across the life cycle. J Clin Psychiatry 65:22-26.
Spencer T, Biederman J, Wilens T, Faraone S, Prince J, Gerard K, et al (2001): Efficacy of a Mixed Amphetamine Salts Compound in Adults With Attention-Deficit/Hyperactivity Disorder. Arch Gen Psychiatry 58:775-782.
Spencer T, Biederman J, Wilens T, Prince J, Hatch M, Jones J, et al (1998): Effectiveness and tolerability of tomoxetine in adults with attention deficit hyperactivity disorder. Am J Psychiatry 155:693-695.
Wilens T, Biederman J, Spencer T, Prince J (1995): Pharmacotherapy of adult attention deficit/hyperactivity disorder: A review. J Clin Psychopharmacol15:270-279.
Wilens T, Faraone SV, Biederman J (2004): Attention-Deficit/Hyperactivity Disorder in Adults. JAMA 292:619-23.
Wilens T E, Biederman J, Spencer T J, Bostic J, Prince J, Monuteaux M C, et al (1999): A pilot controlled clinical trial of ABT-418, a cholinergic agonist, in the treatment of adults with attention deficit hyperactivity disorder. Am J Psychiatry 156:1931-7.

What is claimed is:

1. A method of identifying a neuronal nicotinic receptor ligand, comprising the steps of:
(a) assessing a compound for selective binding to α4β2 neuronal nicotinic receptor subtype;
(b) assessing a compound for ability to stimulate ion channel flux into a cell expressing α4β2, α3β4, or α3β2 neuronal nicotinic receptor subtypes; and
(c) identifying a compound selected for α4β2 neuronal nicotinic receptor subtype that demonstrates weak ability to stimulate ion channel flux into the cell expressing α4β2, α3β4, or α3β2 neuronal nicotinic receptor subtypes that is not a neuronal nicotinic receptor antagonist.

2. The method of claim 1, wherein the compound is assessed for selective binding by [³H]-cytisine assay.

3. The method of claim 1, wherein the compound demonstrates less than 30 nM binding affinity when measured by [³H]-cytisine binding.

4. The method of claim 1, wherein the compound is assessed for ability to stimulate ion channel flux by measuring ⁸⁶Rb⁺ flux into cells of IMR-32 human neuroblastoma clonal cell line.

5. The method of claim 1, wherein the compound demonstrates less than 40% maximal agonist efficacy when measuring ⁸⁶Rb⁺ flux in cells of IMR-32 human neuroblastoma clonal cell line.

6. The method of claim 1, wherein the compound identified has the structure:

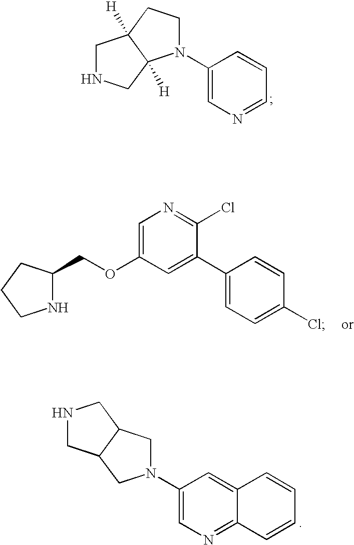

7. A method for treating a mammal having a condition or disorder where modulation of nicotinic acetylcholine receptor activity is of therapeutic benefit, the method comprising administering to a subject having or subsceptible to said condition or disorder with a therapeutically effective amount of a compound demonstrating selective binding for α4β2 neuronal nicotinic receptor subtype and weak agonist activity in cells expressing α4β2, α3β4, or α3β2 neuronal nicotinic receptor subtypes, except neuronal nicotinic receptor antagonists.

8. The method of claim 7, wherein the compound is assessed for ability to stimulate ion channel flux by measuring ⁸⁶Rb⁺ into cells of IMR-32 human neuroblastoma clonal cell line.

9. The method of claim 7, wherein the compound is

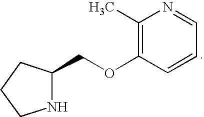

10. The method of claim 7, wherein the compound is:

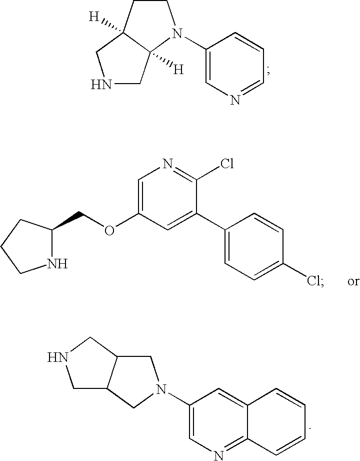

11. The method of claim 7, wherein the condition or disorder is characterized by neuropsychological and cognitive dysfunction.

12. The method of claim 7, wherein the condition or disorder is Alzheimer's disease, bipolar disorder, schizophrenia, or schizoaffective disorder.

13. The method of claim 7, wherein the condition or disorder is Alzheimer's disease.

14. The method of claim 7, wherein the mammal or subject demonstrates low incidence of cardiovascular or gastrointestinal irregularities, or both.

15. The method of claim 7, further comprising administering the compound at a level that is ten-fold the neuronal nicotinic binding Kᵢ value obtained when measuring selective α4β2 neuronal nicotinic receptor subtype binding.

16. A method of using ABT-089 human clinical data to obtain regulatory authorization, comprising the steps of:
(a) providing ABT-089 human clinical data to a regulatory agency having authority to assess or regulate, or both, pharmaceutical compounds or products, or both; and
(b) obtaining approval to manufacture or market a desired pharmaceutical compound from the regulatory agency.

17. The method of claim 16, wherein the human clinical data is related to a randomized, double-blind, placebo-controlled multiple dose study.

18. The method of claim 16, further comprising the step of providing a pharmaceutical product related to the approval to manufacture or market a desired pharmceutical compound obtained from the regulatory agency.

19. The method of claim 18, wherein the pharmaceutical product is useful for treating a mammal having a condition where modulation of nicotinic acetylcholine receptor activity is of therapeutic benefit, wherein the condition is Alzheimer's disease, bipolar disorder, schizophrenia, or schizoaffective disorder.
